(19) United States
(12) Patent Application Publication
(10) Pub. No.: US 2012/0031398 A1
(43) Pub. Date: Feb. 9, 2012

(54) NASAL SPRAY DEVICE

(75) Inventors: Robert L. Berger, Bound Brook, NJ (US); Michael L. Turini, Cranford, NJ (US); Peter A. Basile, Bloomsbury, NJ (US); Stephen Miggels, Wyckoff, NJ (US); Henry J. Mack, Phillipsburg, NJ (US)

(73) Assignee: SCHERING CORPORATION

(21) Appl. No.: 13/127,418

(22) PCT Filed: Nov. 3, 2009
(86) PCT No.: PCT/US09/63071

§ 371 Date: Oct. 11, 2011

Related U.S. Application Data
(60) Provisional application No. 61/110,906, filed on Nov. 3, 2008.
(60) Provisional application No. 61/110,912, filed on Nov. 3, 2008.

(51) Int. Cl. A61M 11/02; A61M 11/00

(52) U.S. Cl. 128/200.21; 128/200.14

(57) ABSTRACT

A delivery device (10) for the delivery of at least one dose of a composition into a nasal cavity, the device has a discharge member (12) formed for insertion into a nasal cavity, a discharge aperture (20) being formed therethrough and a body (14) containing a reservoir (24) of at least one dose, the body is elongated and generally extending along a longitudinal axis (16), wherein the discharge member (12) extends along a secondary axis (18), the secondary axis (18) intersecting with, and being disposed transversely to the longitudinal axis (16).

### FIELD OF THE INVENTION

This invention relates to devices for delivering at least one dose of a composition to the nasal cavity.

### BACKGROUND

Nasal spray inhalers are known in the prior art. A nasal spray inhaler may include an elongated discharge member formed for insertion into a nasal cavity. A reservoir is coupled to the discharge member with spray actuation being achieved by squeezing the discharge member towards the reservoir. Such devices suffer from several deficiencies. First, the discharge member has a substantially long length due to the necessity for sufficient insertion into the nasal cavity and accommodation of a user's fingers thereabout. In addition, it is difficult to consistently locate the tip of the discharge member, having the spray orifice, at a position within the nostril where it is at an appropriate distance and is in axial alignment with the nasal valve to ensure delivery of the drug to the posterior nasal cavity when a patients fingers are placed atop of the gripping surface of the discharge member and beneath their nose. During squeezing motion necessary to actuate the device, the tip of the discharge member may move in reaction to the patient input motion which can result in misalignment and less efficient dose administration. Additionally, these designs can be difficult and unsanitary to use when administering a drug to others, such as in the case when a parent administers a spray to a child.

Thus, there is a need for a new nasal spray device that overcomes these shortcomings.

### SUMMARY OF THE INVENTION

Several embodiments of the present invention provide for a delivery device for the delivery of at least one dose of a composition into a nasal cavity. The composition may be a liquid or powder composition. The delivery device includes a discharge member formed for insertion into a nasal cavity, a discharge aperture being formed therethrough. The delivery device may further include a body containing a reservoir of at least one dose of a composition, such as a liquid or powder composition. The body is elongated and generally extending along a longitudinal axis. Further, the discharge member extends along a secondary axis. The secondary axis intersects with, and is disposed transversely to, the longitudinal axis.

Advantageously, a delivery device is provided having a discharge member which is not axially aligned with the body of the delivery device. In this manner, the body may be maintained away from the face of a user during dose administration. In addition, a relatively short discharge member may be provided.

Other embodiments provide a delivery device for the delivery of at least one dose of a composition into a nasal cavity, the device including a discharge member formed for insertion into a nasal cavity, a discharge aperture being formed therethrough; a stop surface defined at least partially about the discharge member, the stop surface extending radially outwardly from the discharge member, the stop surface limiting the extent the discharge member may be inserted into a nasal cavity; and, a body containing a reservoir of at least one dose, the body being elongated and generally extending along a longitudinal axis, wherein the discharge member extends along a secondary axis, the secondary axis intersecting with, and being disposed transversely to, the longitudinal axis.

Various embodiments of the present invention provide a delivery device for the delivery of at least one dose of a composition into a nasal cavity, the device including a discharge member formed for insertion into a nasal cavity, a discharge aperture being formed therethrough; and, a body containing a reservoir of at least one dose, the body being elongated and generally extending along a longitudinal axis, wherein the discharge member extends along a secondary axis, the secondary axis intersecting with, and being disposed transversely to, the longitudinal axis; wherein the reservoir is elongated and extends along the longitudinal axis, and; wherein a portion of the reservoir extends transversely away from the longitudinal axis to define a well for collecting a liquid or powder composition gravitationally below the longitudinal axis, during use of the delivery device.

Other embodiments of the present invention provide for a drug product comprising a delivery device and a composition comprising at least one active pharmaceutical agent. Suitable active pharmaceutical agents include but are not limited to mometasone furoate, such as mometasone furoate monohydrate or mometasone furoate anhydrous, fluticasone furoate, fluticasone propionate, budesonide, triamcinolone, ciclesonide, oxymetazoline, azelastine, olopatadine, montelukast and combinations thereof or pharmaceutically acceptable salts thereof.

Various embodiments of the present invention provide a delivery device for the delivery of at least one dose of a composition into a nasal cavity, the device including a discharge member formed for insertion into a nasal cavity, a discharge aperture being formed therethrough; and, a body containing a reservoir of at least one dose, the body being elongated and generally extending along a longitudinal axis; a trigger for causing delivery of at least one dose from the reservoir through the discharge aperture, the trigger being located on the body and spaced from the discharge member; wherein the discharge member extends along a secondary axis, the secondary axis intersecting with, and being disposed transversely to, the longitudinal axis; and wherein the trigger is configured to have a force applied thereto for actuation, the force being applied in a direction generally oblique to the longitudinal axis. The secondary axis may subtend an acute angle with the longitudinal axis. The acute angle may be in the range from about 15° to about 45°. The force for actuation may be applied to the trigger in a direction generally parallel to the secondary axis. The discharge member may terminate at a free end with the discharge aperture being formed through the free end. The discharge aperture may be formed at the furthest most location on the discharge member from the body. A pump for delivering the at least one dose from the reservoir and through the discharge aperture may be included. The trigger may be pivotally attached to the body. The trigger may extend along a central axis, the central axis subtends an acute angle with the longitudinal axis. The acute angle may be in the range from about 15° to about 45°. The force for actuation may be applied to the trigger in a direction generally perpendicular to the central axis. Other embodiments provide a drug product with the device and a composition comprising at least one active pharmaceutical agent. Suitable at least active pharmaceutical agents include mometasone furoate, such as mometasone furoate monohydrate or mometasone furoate anhydrous.

These and other features of the invention will be better understood through a study of the following detailed description and accompanying drawings.

### BRIEF DESCRIPTION OF THE DRAWINGS

FIGS. 1-4 depict a delivery device formed in accordance with the various embodiments of the present invention;

FIG. 5 shows schematically use of a delivery device formed in accordance with the various embodiments of the present invention;

FIG. 5A is a schematic showing application of force to actuate a delivery device formed in accordance with the various embodiments of the present invention;

FIGS. 6 and 7 are partial views of a delivery device formed in accordance with the various embodiments of the present invention;

FIG. 8 is an exploded view of a delivery device formed in accordance with the various embodiments of the present invention;

FIGS. 9-11 show different stages of actuation of a delivery device formed in accordance with the various embodiments of the present invention; and,

FIGS. 12 and 13 depict a reservoir usable with the various embodiments of the present invention.

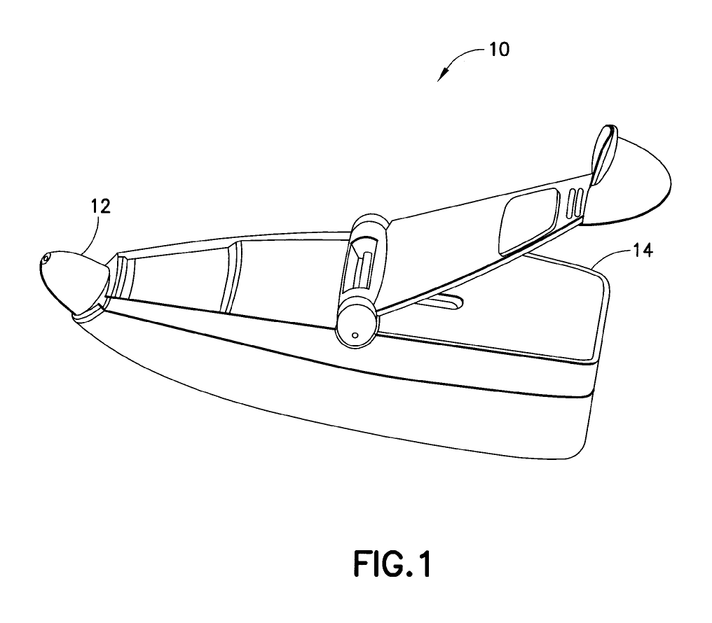

### DETAILED DESCRIPTION

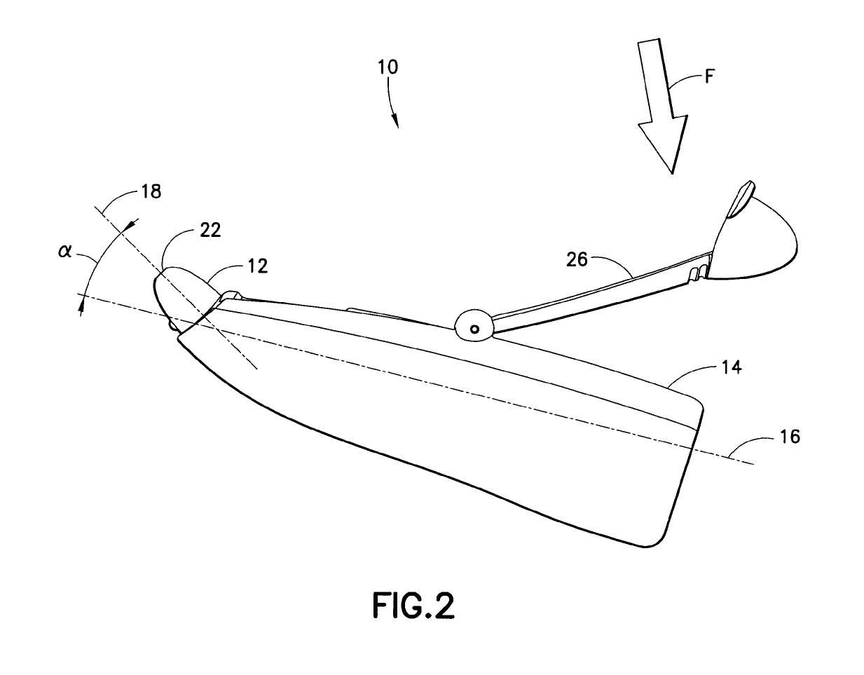

As shown generally in the figures, several embodiments of the present invention provide a delivery device 10 for the delivery of at least one dose of a composition, such as a liquid or powder composition, into a nasal cavity of a user. The delivery device 10 generally includes a discharge member 12 and a body 14. With reference to FIG. 2, the body 14 is elongated and extends along a longitudinal axis 16. The discharge member 12 extends along a secondary axis 18. The secondary axis 18 intersects with, and is disposed transversely to, the longitudinal axis 16. With this arrangement, the discharge member 12 is not axially aligned with the longitudinal axis 16. As shown in FIG. 2, an angle α may be subtended by the longitudinal axis 16 and the secondary axis 18. The angle α may be 90° or an acute angle, such as in the range from about 15° to about 45°, or about 30°.

The discharge member 12 is formed for insertion into a nasal cavity. In several embodiments, the discharge member 12 is generally bullet-shaped. The discharge member 12 may have a maximum length of 0.75″ and a maximum diameter of 0.5″. A discharge aperture 20 is formed through the discharge member 12 to permit at least one dose of a composition, such as a liquid or powder composition, to be administered from the delivery device 10. The discharge member 12 extends from the body 14 to have a free end 22. The discharge aperture 20 may be formed through the free end 22 (e.g., centrally through the free end 22), or the discharge aperture 20 may be formed at the furthestmost location on the discharge member 12 from the body 14. The discharge member 12 may be formed of various materials suitable for insertion into a nasal cavity.

The discharge member 12 may be immovably fixed to the body 14. The discharge member 12 may be unitarily formed with the body 14 or attached thereto using any known technique.

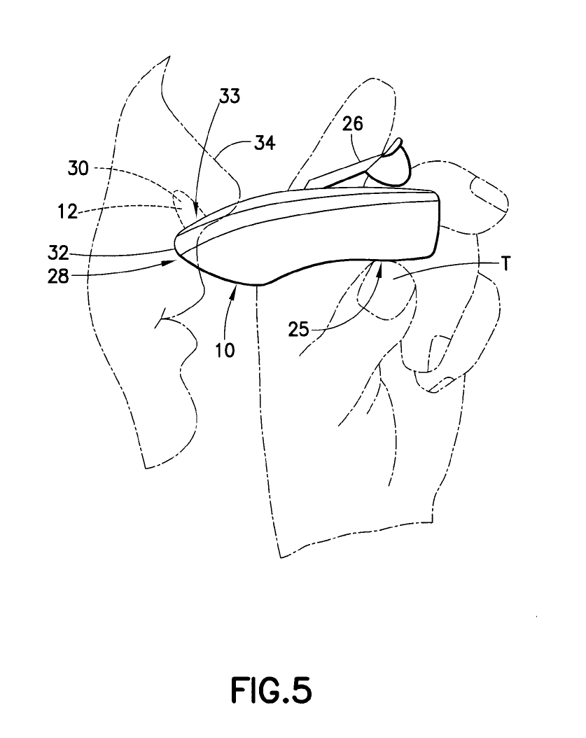

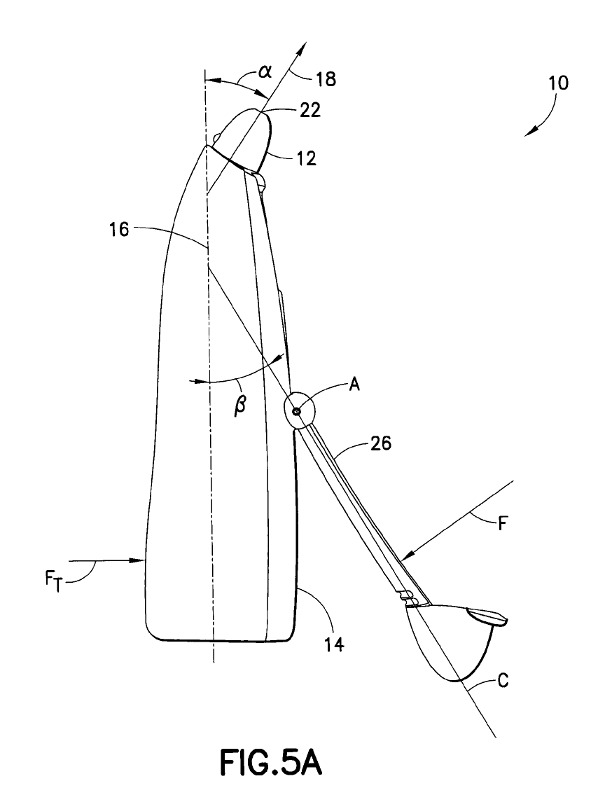

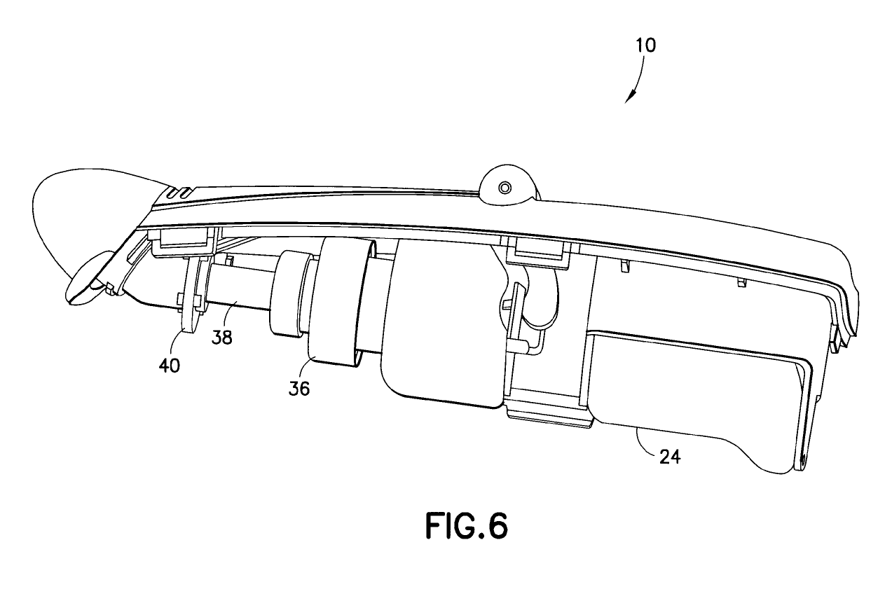

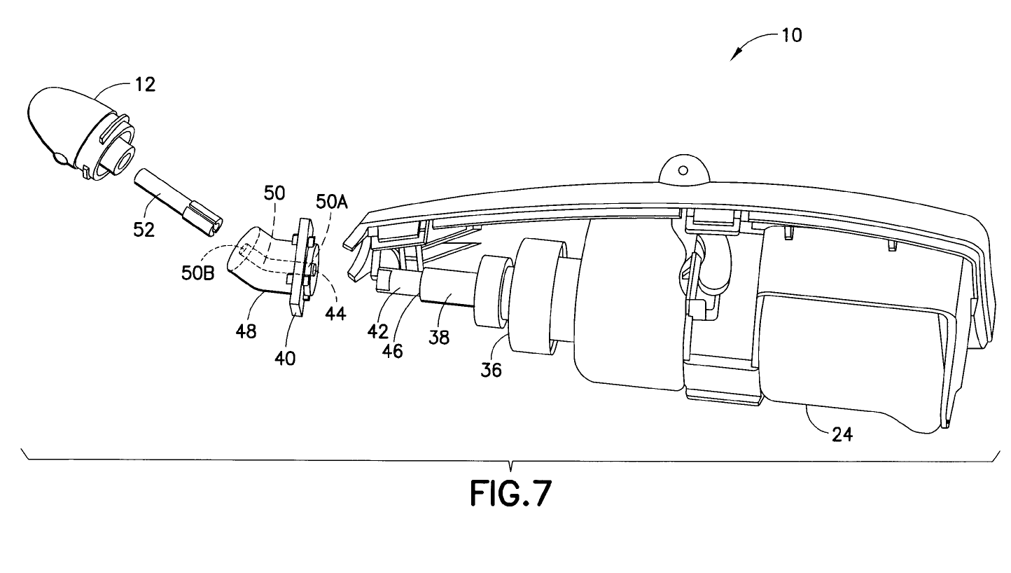

The body 14 contains a reservoir 24 (best shown in FIGS. 6 and 7) which houses a at least one dose to be administered by the delivery device 10. In addition, a trigger 26 may be provided on the body 14 formed to cause delivery of at least one dose of a composition from the reservoir 24 through the discharge aperture 20. The trigger 26 may be located on the body 14 and spaced from the discharge member 12. The trigger 26 may be adapted to be actuated with force being applied thereto in a direction transverse to the longitudinal axis 16, as shown schematically by arrow F in FIG. 2. With this arrangement, as shown in FIG. 5, the delivery device 10 may be pressed against the upper lip 28 of a user's face with the discharge member 12 being nested in the user's nasal cavity 30. With the trigger 26 being spaced from the discharge member 12, operation of the delivery device 10 may be achieved without interference of the person's face and may be one-handed. The trigger 26 may be located on an upper side of the body 14 so as to face upwardly during use, as shown in FIG. 5. In addition, a front end 32 of the delivery device 10, as pressed up against the upper lip 28, acts as a resting surface during actuation. With this arrangement, the subject invention provides for a stable positioning of the delivery device 10 during actuation and an effective alignment of the discharge aperture 20 within the nasal cavity 30.

With reference to FIG. 5, the delivery device 10 may be formed such that a thumb T of a user may be located to counteract any movement to the discharge member 12 in reaction to the force or movement of trigger 26 in causing activation thereof. To facilitate proper location of the thumb T, an identifiable area 25 may be defined on the body 14 located so as to counteract any force or moment applied to the trigger 26. The identifiable area 25 may be: of a different material from the body 14; and/or, a contoured, textured and/or colored area on the body 14. Instructions corresponding to the delivery device 10 may also be relied upon in showing desired placement of the thumb T.

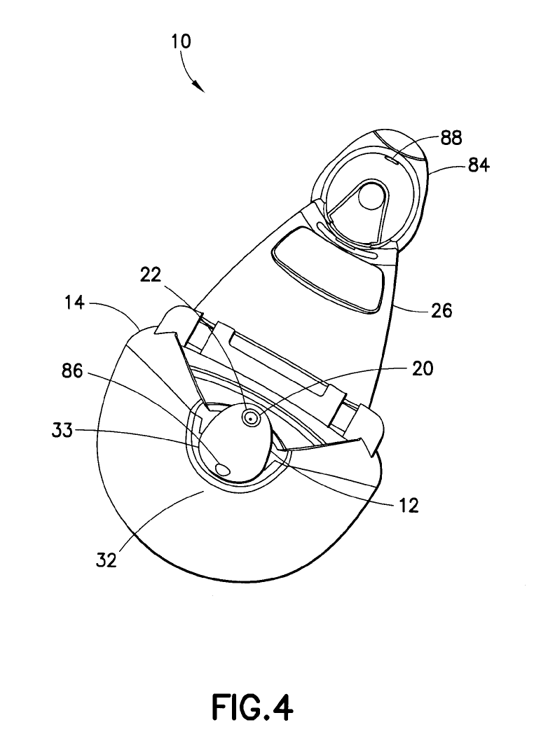

With reference to FIG. 4, a continuous or discontinuous stop surface 33 may be defined at least partially about the base of the discharge member 12. The stop surface 33 may be annular and encircles the discharge member 12. As shown in FIG. 4, the stop surface 33 extends radially outwardly from the discharge member 12. With reference to FIG. 5, the stop surface 33 acts to press against a user's nose 34 in limiting the depth of insertion of the discharge member 12. The ability to limit depth insertion allows for the positioning of the discharge aperture 20 relative to the user's anatomy. The extent of insertion of a nasal delivery device into a user's nose affects deposition of the delivered dose due to the distance a dose is delivered within the nasal cavity. The stop surface 33 provides for more accurate placement of the discharge aperture 20.

In addition, the front end 32, may be flattened or truncated or provided with little curvature adjacent to the stop surface 33 to provide a stable alignment surface for acting against the upper lip 28 in angularly aligning the nasal valve of device 10 with the nose 34 of the user. The front end 32 minimizes rocking or other angular or rotational variation, including during application of actuation force to the trigger 26.

The angled discharge member 12 relative to the body 14 of the delivery device 10 locates a patient's hands, during placement and actuation, away from their face. This locates a patient's hands away from their nose where it can come into contact with nasal discharge. The discharge member 12 is designed to be placed against the face above the upper lip 28 and beneath the nasal cavity 30, with the nose as a reference surface to assist in axially locating the spray orifice of the discharge member with the nasal valve when placed within the nasal cavity 30. Accordingly, the underside of the nose provides a depth locator when the discharge member 12 is placed upward within the nasal cavity 30. The length of the discharge member 12 is short as compared to the prior art, to place the discharge aperture 20 at a proper distance from the nasal valve when placed within the nasal cavity 30. Furthermore, the trigger 26 and the body 14 of the device 10 have been designed to generally align the forces and reaction forces in opposing fashion during actuation, minimizing any reaction moments moving the discharge member 12 during actuation. The combination of these three features (hands-away actuation; depth location; general orientation of actuation and counter forces) increases the likelihood of proper placement during administration and allows for easier administration to users.

With reference to FIG. 5A, the force F may be applied to the trigger 26 in a direction oblique to the longitudinal axis 16. An acute angle β may be subtended between a central axis C of the trigger 26 and the longitudinal axis 16 with the trigger 26 being in a ready state for actuation. As discussed below, the trigger 26 may be a lever-type actuator, with the force F generating moment to cause actuation of the trigger 26. Taking force F being applied generally perpendicularly to the trigger 26, as taken relative to the central axis C, the force F is disposed obliquely relative to the longitudinal axis 16. In addition, the angles α and β may be equal. With this arrangement, the force F may be parallel to the secondary axis 18. The secondary axis 18 may coincide with the path of fluid delivered by the delivery device 10. Accordingly, the force F may be parallel to the path of fluid delivery. The angle β may be in the range from about 15° to about 45°, or about 30°. With the parallel arrangement of the force F and the path of fluid delivery, moment created by the application of the force F is counteracted by the force created by delivery of the fluid. In addition, as discussed above, the thumb T provides a counteracting force to offset the force F applied to the trigger 26. With reference to FIG. 5A, force F_{T} generated by the thumb T is shown. As shown in FIG. 5A, the trigger 26 pivots about axis A. The forces F and F_{T} may be applied generally at equal distances from the axis A so as to generate counteracting movements thereabout.

As will be recognized by those skilled in the art, the delivery device 10 may be self-administered or administered by a third party to a user. For example, a parent or medical practitioner may administer at least one dose of a composition by the delivery device 10 to a child or elderly patient.

Any configuration for delivering a dose of a composition from the reservoir 24 through the discharge aperture 20 may be utilized, particularly a trigger-activated configuration. By way of non-limiting example, a pump 36 may be provided to deliver a dose of a composition from the reservoir 24 through the discharge aperture 20. The pump 36 may be provided with a discharge tube 38, as best shown in FIGS. 7 and 13. The pump 36 may be configured in the same manner as any pump provided with a metered dose nasal spray, where compression of the discharge tube 38 into the reservoir 24 results in a dose of composition being discharged through the discharge tube 38. The reservoir 24 may be the canister of a metered dose nasal spray. With respect to the subject invention, the discharge tube 38 may be fixed to a stop block 40 disposed in the body 14. The discharge tube 38 may include a thinned region 42 formed to be received within a corresponding aperture 44 in the stop block 40. A shoulder 46 is defined on the discharge tube 38 about the thinned region 42 which is formed larger than the aperture 44. With the stop block 40 being fixed within the body 14, forward translation of the reservoir 24 causes interengagement between the shoulder 46 and the stop block 40. With further forward translation of the reservoir 24, the shoulder 46 restricts movement of the discharge tube 38. As the reservoir 24 is further translated, and the discharge tube 38 is restricted from forward movement, the discharge tube 38 is compressed into the pump 36 resulting in the pump 36 being actuated.

To permit delivery of at least one dose of a composition from the discharge tube 38 through the discharge aperture 20, a change of direction element 48 is provided having a channel 50 formed therethrough to register with the aperture 44. The channel 50, in turn, is in registration with the discharge aperture 20 to permit delivery of the fluid. The channel 50 includes first and second sections 50A, 50B angularly off-set from one another. The sections 50A, 50B may be angularly off-set the angle α as described above. The stop block 40 and the change of direction element 48 may be formed separately and joined or formed unitarily as a single component. A nozzle 52 may be disposed along the fluid path between the stop block 40 and the discharge aperture 20 to control the resulting spray pattern of the administered dose. The nozzle 52 may be disposed to define the discharge aperture 20.

With the arrangement described above, the trigger 26 may be coupled to the reservoir 24 to cause forward translation thereof upon the trigger 26 being actuated as shown in FIG. 2. After dose administration, and as known in the prior art, with force being removed from the trigger 26, the pump 36 may be provided with a return mechanism, such as a return spring, to urge the reservoir 24 to a ready position for further dose administration.

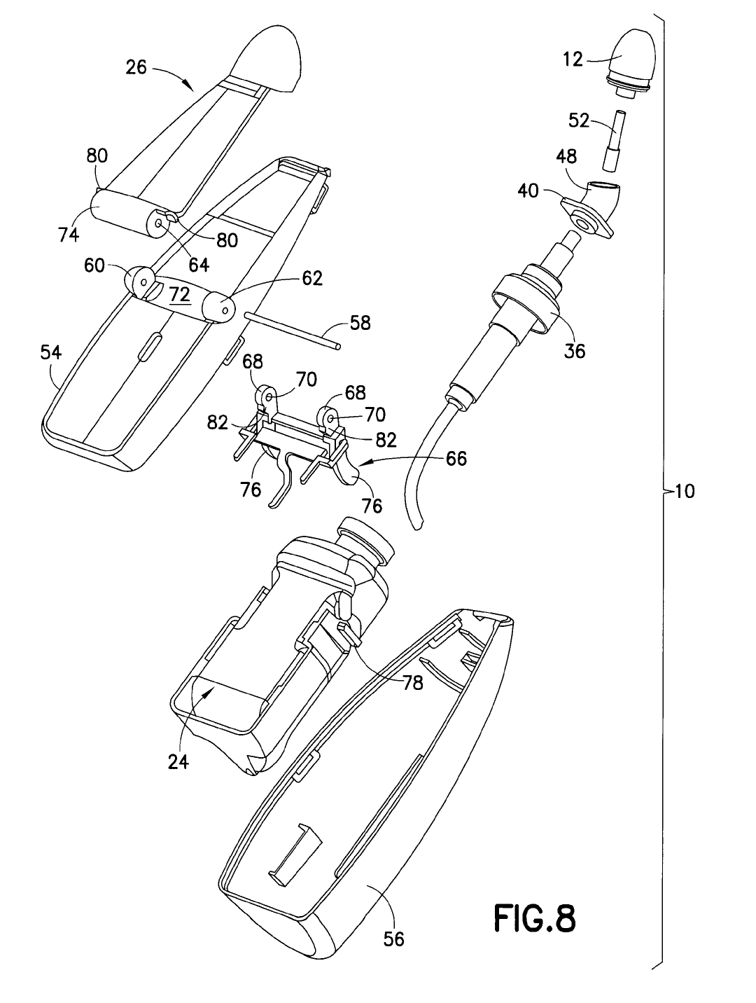

With reference to FIG. 8, a configuration usable with the subject invention is shown for actuating the pump 36. As shown in FIG. 8, the body 14 may be formed from first and second components 54, 56 which may constitute upper and lower housing components, respectively. The trigger 26 may be provided in the form of a lever which is pivotally mounted to the body 14, such as to the first component 54. The pivotal mounting may be defined by a pin 58 held on the body 14 by end pin holders 60, 62. The trigger 26 may include passage 64 which is formed to receive the pin 58 with the trigger 26 being configured to rotate about the pin 58 relative to the body 14. The pin 58 may define the axis A discussed above.

An actuator 66 is provided to transmit force from the trigger 26 to the reservoir 24 to cause forward translation thereof. The actuator 66 includes at least one mounting block 68 or two of the mounting blocks 68, each having an aperture 70 formed therethrough. The aperture(s) 70 are formed to receive the pin 58. The actuator 66 is mounted into the body 14 with the one or more mounting blocks 68 extending through an opening 72 formed in the body 14 with the pin 58 passing through the one or more apertures 70. The passage 64 of the trigger 26 is defined in an end member 74. The one or more mounting blocks 68 are positioned to be adjacent to one or both ends of the end member 74.

The actuator 66 also includes one or more actuating arms 76 formed and positioned to engage against corresponding actuating surfaces 78 on the reservoir 24. The actuator may 66 have a yoke shape with two of the actuating arms 76 being provided, the actuating arms 76 being spaced apart to straddle the reservoir 24. With the actuating arm(s) 76 being forced forwardly, force is applied against the actuating surface(s) 78 resulting in forward translation of the reservoir 24.

To transmit force from the trigger 26 to the actuator 66, one or both ends of the end member 74 is provided with a protrusion 80 of limited circumferential length. Also, a notch 82 is formed in each of the mounting blocks 68 configured to be engaged by one of the protrusions 80.

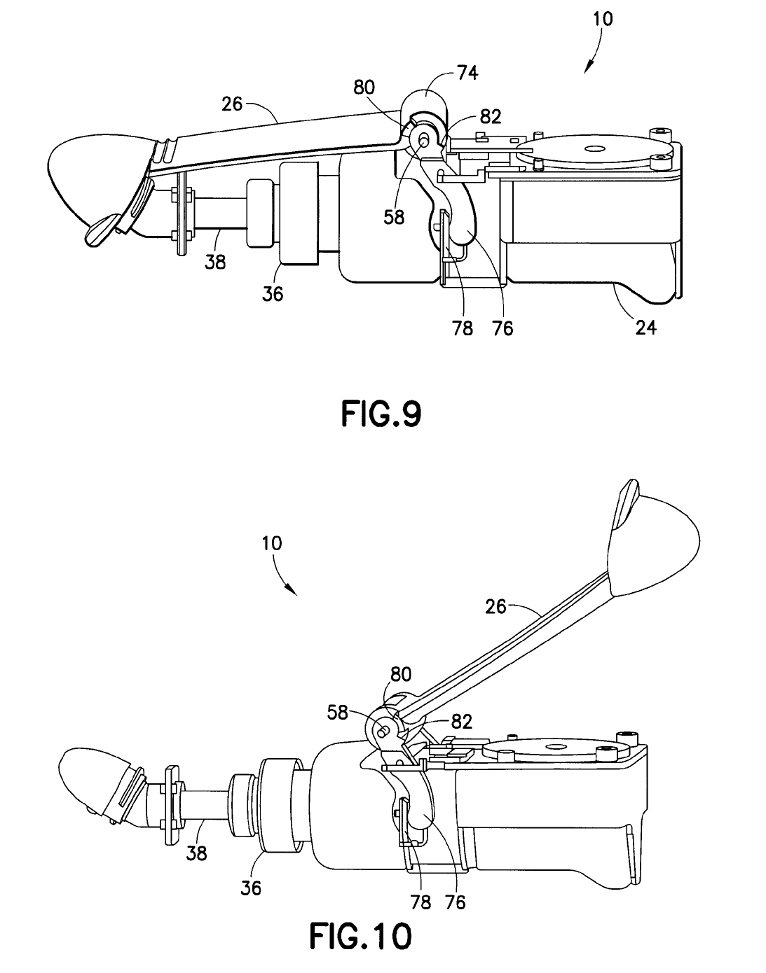

With reference to FIGS. 9-11, an actuation of the delivery device 10 is shown in different phases. FIG. 9 shows the delivery device in an inactive state. In this state, return force generated by the pump 36 maintains the reservoir 24 in a rearmost state relative to the body 14.

With reference to FIG. 10, the trigger 26 is shown in a ready-to-use state, where the protrusions 80 are in contact with the notches 82. As shown in FIG. 9, in an inactive state, the protrusions 80 are spaced from the notches 82. The trigger 26 is rotated from the inactive to ready-to-use states. Rotation of the trigger 26 to the ready-to-use state is done freely without any contact between the protrusions 80 and the notches 82. The circumferential length of the protrusions 80 and the shape and positioning of the notches 82 defines the radial position of the trigger 26 for the ready-to-use state. More specifically, the circumferential length of the protrusions 80 and the shape and positioning of the notches 82 defines the angle β.

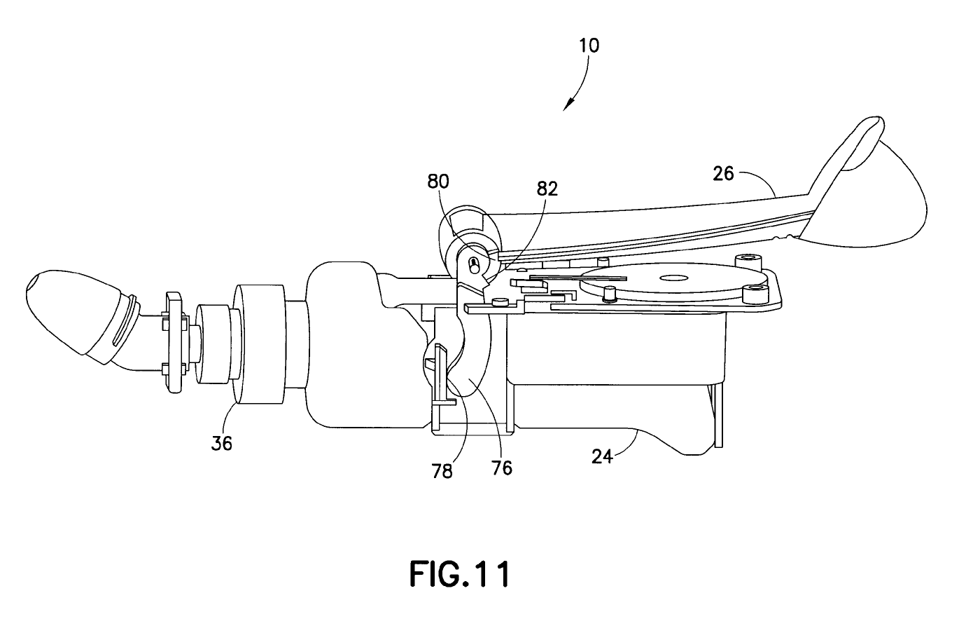

To cause actuation, force is applied, to the trigger 26, as shown in FIG. 2, resulting in depression of the trigger 26 as shown in FIG. 11. Under force, the trigger 26 further rotates with rotation of the trigger 26 from the position shown in FIG. 10 to the position shown in FIG. 11 causing the protrusions 80 to urge the notches 82 forwardly. With the actuator 66 being pivotally mounted to the pin 58, the protrusions 80 cause the notches 82 to rotate about the pin 58. Rotation of the notches 82 results in pivotal movement of the one or more of the actuating arm(s) 86. As a result, the actuating arms 86 press against the actuating surfaces 78 and cause forward translation of the reservoir 24. The reservoir 24 will be urged sufficiently forwardly to cause proper actuation of the pump 36 and administration of a dose of a composition. The discharge tube 38 is visible in the states shown in FIGS. 9 and 10, but not visible in the state shown in FIG. 11. In an actuated state, the discharge tube 38 is compressed into the pump 36 for actuation.

With force being removed from the trigger 26 after dose administration, return force from the pump 36 urges the reservoir 24 rearwardly to the inactive state, such as that shown in FIG. 9. In addition, the trigger 26 is returned to the ready-to-use state as shown in FIG. 10. Any force of actuation required to actuate the trigger 26 must be sufficiently great to overcome the bias return force of the pump 36.

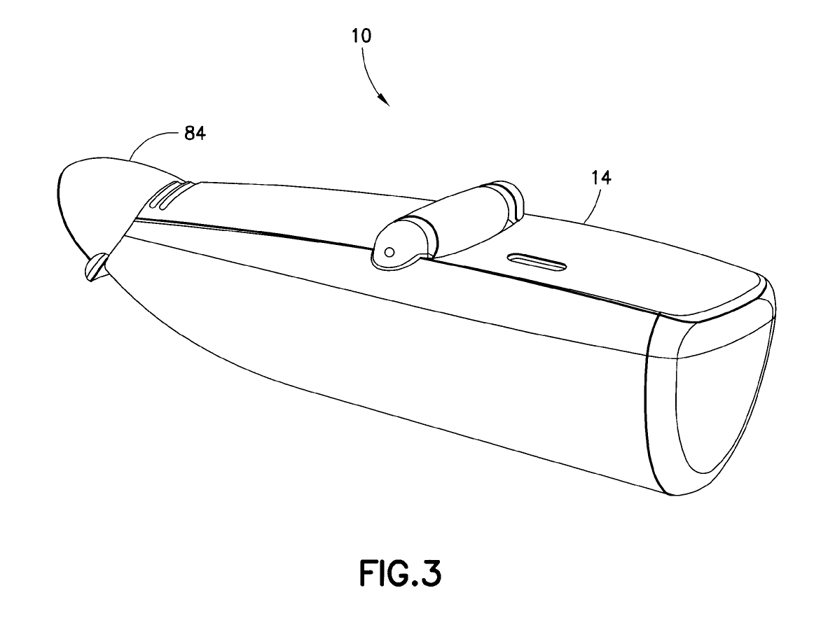

A cap 84 may be provided to cover the discharge aperture 20 (FIGS. 3 and 9). A releasable locking arrangement between the discharge member 12 and the cap 84 may be provided. As shown in FIG. 4, the releasable locking arrangement may be a cooperating locking detent 86 and locking aperture 88 formed on the discharge member 12 and the cap 84. FIG. 4 shows the locking detent 86 being formed on the discharge member 12, with the locking aperture 88 being formed on the cap 84. The reverse configuration may also be provided. The cap 84 may also be provided on the trigger 26 or at the end of the trigger 26. In an inactive state, as shown in FIG. 9, the trigger 26 may be folded down with the cap 84 covering the discharge aperture 20.

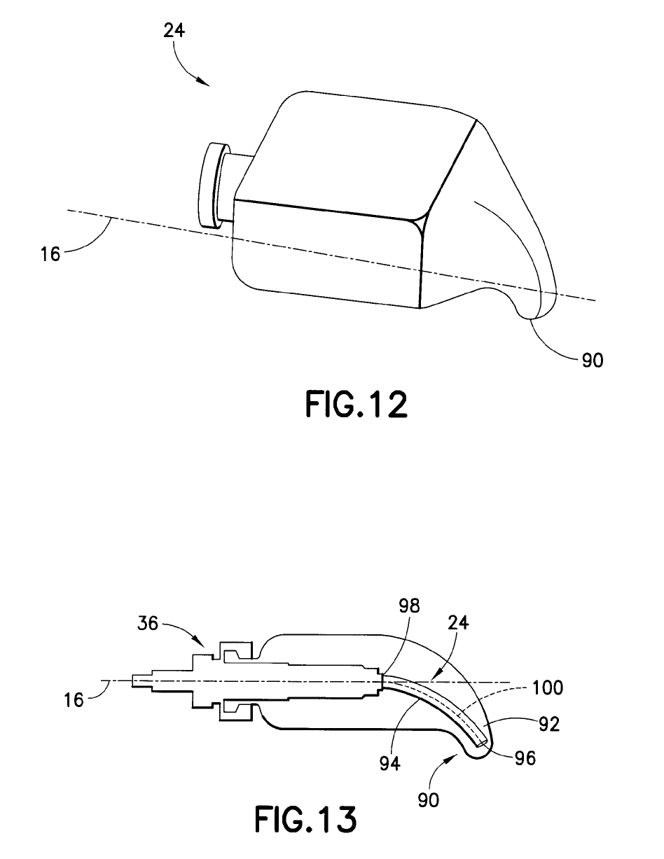

With reference to FIGS. 12 and 13, the reservoir 24 may be elongated and formed to extend along the longitudinal axis 16. With the delivery device 10 being generally in a horizontal position during actuation, efficient consumption of the fluid stored within the reservoir 24 may be of concern. As shown in FIGS. 12 and 13, a portion 90 of the reservoir 24 may extend transversely away from the longitudinal axis 16. The portion 90 may define a well 92 for collecting a liquid or powder composition gravitationally below the longitudinal axis 16. With this arrangement, low levels of a liquid or powder composition in the reservoir 24 may be collected within the well 92. A dip tube 94 may be further provided having first and second ends 96, 98 and a lumen 100 extending therebetween. The first end 96 may be positioned in the well 92, or at a bottom-most section of the well 92 (furthermost section from the longitudinal axis 16), with the second end 98 of the dip tube 94 being in communication with the pump 36. As arranged, the lumen 100 communicates the well 92 with the pump 36. Low levels of a liquid or powder composition may thus be extracted from the well 92 for delivery to a user.

The descriptions of the embodiments of the invention have been presented for purpose of illustration and description. They are not intended to be exhaustive or to limit the invention to the precise forms disclosed, and obviously many modifications and variations are possible in light of the above teaching.

1. A delivery device for the delivery of at least one dose of a composition into a nasal cavity, the device comprising:
a discharge member formed for insertion into a nasal cavity, a discharge aperture being formed therethrough; and,
a body containing a reservoir of at least one dose, the body being elongated and generally extending along a longitudinal axis, wherein the discharge member extends along a secondary axis, the secondary axis intersecting with, and being disposed transversely to, the longitudinal axis.

2. A delivery device as in claim 1, wherein the secondary axis subtends an acute angle with the longitudinal axis.

3. A delivery device as in claim 1, wherein the secondary axis is generally perpendicular to the longitudinal axis.

4. A delivery device as in claim 1, further comprising a trigger for causing delivery of at least one dose from the reservoir through the discharge aperture, the trigger being located on the body and spaced from the discharge member.

5. A delivery device as in claim 4, wherein the trigger is adapted to be actuable with force being applied thereto in a direction transverse to the longitudinal axis.

6. A delivery device as in claim 1, wherein the discharge member terminates at a free end, the discharge aperture being formed through the free end.

7. A delivery device as in claim 6, wherein the discharge aperture is formed at the furthestmost location on the discharge member from the body.

8. A delivery device as in claim 1, wherein the reservoir is elongated and extends along the longitudinal axis.

9. A delivery device as in claim 8, wherein a portion of the reservoir extends transversely away from the longitudinal axis.

10. A delivery device as in claim 9, wherein the portion defines a well for collecting the composition gravitationally below the longitudinal axis during use of the delivery device.

11. A delivery device as in claim 10, further comprising a pump for delivering the at least one dose from the reservoir and through the discharge aperture.

12. A delivery device as in claim 11, further comprising a dip tube having first and second ends with a lumen extending therebetween, the first end being positioned in a furthermost section of the well from the longitudinal axis, a second end of the dip tube being in communication with the pump, the lumen communicating the well with the pump.

13. (canceled)

14. A delivery device as in claim 11, further comprising a trigger for causing actuation of the pump to cause delivery of the at least one dose from the reservoir through the discharge aperture.

15. (canceled)

16. (canceled)

17. (canceled)

18. A delivery device for the delivery of at least one dose of a composition into a nasal cavity, the device comprising:
a discharge member formed for insertion into a nasal cavity, a discharge aperture being formed therethrough;
a stop surface defined at least partially about the discharge member, the stop surface extending radially outwardly from the discharge member, the stop surface limiting the extent the discharge member may be inserted into a nasal cavity; and,
a body containing a reservoir of at least one dose, the body being elongated and generally extending along a longitudinal axis, wherein the discharge member extends along a secondary axis, the secondary axis intersecting with, and being disposed transversely to, the longitudinal axis.

19. (canceled)

20. (canceled)

21. (canceled)

22. (canceled)

23. (canceled)

24. (canceled)

25. (canceled)

26. (canceled)

27. A delivery device for the delivery of at least one dose of composition into a nasal cavity, the device comprising:
a discharge member formed for insertion into a nasal cavity, a discharge aperture being formed therethrough; and,
a body containing a reservoir of at least one dose, the body being elongated and generally extending along a longitudinal axis;
a trigger for causing delivery of at least one dose from the reservoir through the discharge aperture, the trigger being located on the body and spaced from the discharge member;
wherein the discharge member extends along a secondary axis, the secondary axis intersecting with, and being disposed transversely to, the longitudinal axis; and
wherein the trigger is configured to have a force applied thereto for actuation, the force being applied in a direction generally oblique to the longitudinal axis.

28. A delivery device as in claim 27, wherein the secondary axis subtends an acute angle with the longitudinal axis.

29. A delivery device as in claim 28, wherein the acute angle is in the range from about 15° to about 45°.

30. (canceled)

31. (canceled)

32. (canceled)

33. (canceled)

34. A delivery device as in claim 27, wherein the trigger is pivotally attached to the body.

35. A delivery device as in claim 27, wherein the trigger extends along a central axis, the central axis subtends an acute angle with the longitudinal axis.

36. (canceled)

37. (canceled)

38. A drug product comprising the device of claim 27 and a composition comprising at least one active pharmaceutical agent.

39. (canceled)
